(19) United States
(12) Patent Application Publication
(10) Pub. No.: US 2022/0008583 A1
(43) Pub. Date: Jan. 13, 2022

(54) Ultraviolet Sterilization Device

(71) Applicant: Manuel Garcia, Miami, FL (US)

(72) Inventor: Manuel Garcia, Miami, FL (US)

(21) Appl. No.: 17/366,250

(22) Filed: Jul. 2, 2021

Related U.S. Application Data
(60) Provisional application No. 63/048,705, filed on Jul. 7, 2020.

(51) Int. Cl. A61L 2/10; A61L 2/26

(52) U.S. Cl. CPC A61L 2/10; A61L 2/26; A61L 2202/14; A61L 2202/122; A61L 2202/20; A61L 2202/121

(57) ABSTRACT

The present invention relates to an ultraviolet sterilization box that disinfects household items such as food, electronics, wallets, keys, sunglasses, etc., and eradicates said items of germs, viruses, bacteria, and other airborne pathogens. The ultraviolet sterilization box includes a bottom surface and four walls extending vertically from the bottom surface that form an enclosed space to store a plurality of items for disinfection. A lid pivotally connected to one wall is configured to cover the enclosed space and one or more UV-C lamps on the interior surfaces of the walls, bottom surface, and the lid provide UV-C radiation from all directions to maximize the exposure of the articles within the enclosed space for sterilization. The UV sterilization box features a timing circuit that enable the UV-C lamps to automatically turn OFF after a predetermined period of time, or manually using a control button, or through a sensor.

### CROSS-REFERENCE TO RELATED APPLICATION

The present application claims priority to, and the benefit of, U.S. Provisional Application No. 63/048,705, which was filed on Jul. 7, 2020 and is incorporated herein by reference in its entirety.

### FIELD OF THE INVENTION

The present invention relates generally to sterilization devices. More specifically, the present invention relates to an ultraviolet (UV) sterilization device configured to sterilize and disinfect groceries and other delivered items, and eradicates delivered items of germs, viruses, bacteria, and other airborne pathogens. The ultraviolet sterilization device has an enclosed space to store items for sterilization and have a plurality of UV lamps to irradiate the stored items. The UV radiations kills the microbes and disinfects the stored items. The UV lamps can be selectively detached and attached to the inner surfaces of the walls, lids and bottom surface of the device or box. Accordingly, the present disclosure makes specific reference thereto. Nonetheless, it is to be appreciated that aspects of the present invention are also equally applicable to other like applications, devices, and methods of manufacture.

### BACKGROUND OF THE INVENTION

By way of background, individuals are increasingly concerned about personal hygiene, i.e., preventing transmission of germs and viruses from one person to another. Personal items such as wallets, keys and sunglasses used in outdoor environment are susceptible to catching, carrying and transferring pathogens. Similarly, items delivered to a home by delivery personnel are also carriers of pathogens. Moreover, items purchased at a store may be infected with germs, bacteria and viruses. This is increasingly dangerous in the times of pandemics, such as COVID-19, where an infected person can easily infect handled items and thus can cause the disease to spread rapidly.

The items that are purchased by a user are touched and/or handled by tens if not hundreds of individuals without any cleaning between uses. Any pathogen that is on a user's hands can be transmitted to the next user.

Generally, individuals use harmful chemicals for disinfecting items that can expose individuals and cause other health issues. Ultraviolet (UV) light of a particular range of wavelengths, intensities and durations can kill or inhibit growth of microorganisms. Specifically, ultraviolet radiation in the range of 200 nanometer (nm) to 300 nm is effective against airborne and surface bacteria, viruses, yeasts and molds. For most microorganisms, the peak inactivation wavelength is in the range from about 240 nm to about 260 nm. Mercury lamps produce UV light very efficiently at 254 nm, and this wavelength has become the standard UV germicidal light wavelength.

However, no method or device yet employed uses the UV light for disinfection in a way that does not expose individuals to the UV radiation while providing effective disinfection. The sterilization boxes, heretofore known, have reflector surfaces and inadequate UV radiations that are unable to provide effective disinfection. There is a need for a new sterilization device for sanitizing/disinfecting items safely and effectively.

Therefore, there exists a long felt need in the art for a sterilization box that can be used for sterilizing and disinfecting food items, bakery products, currency, keys, watches, sunglasses and other household items. There is also a long felt need in the art for a sterilization box that includes germicidal lamps that can be selectively detached and attached for sterilization of the stored items. Additionally, there is a long felt need in the art for a sterilization device that is lightweight, portable and able to sterilize items safely without posing a threat to any user. There is a long felt need in the art for a sterilization box that eliminates or obviates the use of harmful chemicals for disinfecting and sterilizing items. Moreover, there is a long felt need in the art for a sterilization box that sterilizes and irradiates the non-planar surfaces of articles. Finally, there is a long felt need in the art for a sterilization box that provides a safe, convenient and reliable method to thoroughly sterilize and disinfect items quickly.

The subject matter disclosed and claimed herein, in one embodiment thereof, comprises an ultraviolet sterilization box. More specifically, the ultraviolet sterilization box disinfects household items such as food items, electronic items, wallets, keys, sunglasses and the like. The ultraviolet sterilization box is generally cuboidal shaped and includes an internal chamber defined by a bottom surface and four walls extending vertically from the edges of the bottom surface. The internal chamber can store a plurality of items for disinfection. A lid pivotally-connected to one wall is configured to cover the internal chamber. Further, each of the walls, bottom surface and lid can include one or more UV-C lamps on interior surfaces for providing UV-C radiation from all directions to maximize the exposure of the articles within the internal chamber for sterilization. UV-C or UVC radiation is the highest energy portion of the 1.1V radiation spectrum.

In this manner, the novel ultraviolet sterilization and disinfection box of the present invention accomplishes all of the forgoing objectives, and provides a relatively safe, easy and portable device to allow a user to disinfect all types of personal items using UV-C or other types of UV radiations to kill pathogens. The device of the present invention is also user-friendly, as it ensures that a user is not exposed to UV radiations.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises an ultraviolet sterilization box. The ultraviolet sterilization box is configured for disinfecting household items such as food items, electronic items, wallets, keys, sunglasses and the like. The ultraviolet sterilization box further comprises: an internal chamber defined by a bottom surface and four walls extending vertically from the edges of the bottom surface; the internal chamber stores a plurality of items for disinfection; a lid pivotally-connected to one wall and is configured to cover the internal chamber; each of the walls, the bottom surface and the lid have one or more UV-C lamps on interior surfaces for providing UV-C radiation from all directions to maximize the exposure of the articles within the internal chamber for sterilization. The UV-C lamps are automatically turned off after a predetermined period of time, and can be turned on manually using a control button, or automatically through a sensor.

In a further embodiment of the present invention, the lid can be transparent or translucent allowing a user to view the sterilization process. The box does prevents the UV-C radiation from leaking from the surface of the box and the UV-C lamps are automatically turned off when the lid is opened.

In yet a further embodiment of the present invention, a disinfection box for disinfecting household items is disclosed. The disinfection box includes a lid and a base portion. The base portion includes a bottom surface on which the box is placed on any surface and two side walls, a front wall and a rear wall extending vertically from the edges of the bottom surface. The lid is hinged to a top edge of the rear wall, thereby allowing a pivotal movement of the lid along the hinges to cover an enclosed space formed by the walls and the bottom surface. Each of the walls, bottom surface and lid can include a plurality of germicidal UV-C lamps positioned on interior surfaces to provide exposure of UV-C radiation to the items stored in the enclosed space of the box. The enclosed space can include one or more racks to accommodate items at different levels for providing effective sterilization.

In a further embodiment of the present invention, a remotely-operated sterilization box is disclosed. The sterilization box is configured to disinfect items stored within the box using selectively attachable UV-C lamps. The sterilization box can be wirelessly connected to a remote handheld device, and an instruction to turn on/off the UV-C lamps can be received from the remote handheld device.

In a further embodiment of the present invention, the sterilization box can provide a visual and/or audible alarm when the disinfection process has been completed. A pre-configured timer value can be set in a timing circuit, thereby allowing the UV-C lamps to be active for the pre-configured timer value.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one potential embodiment of an ultraviolet sterilization box of the present invention in accordance with the disclosed architecture;

FIG. 2 illustrates a block diagram showing exemplary internal components of one potential embodiment of the ultraviolet sterilization box of the present invention in accordance with the disclosed architecture;

FIG. 3 illustrates a perspective view of items stored within one potential embodiment of an ultraviolet sterilization box of the present invention for sterilization in accordance with the disclosed architecture;

FIG. 4 illustrates a perspective view of one potential embodiment of the ultraviolet sterilization box of the present invention in a closed position with stored items for disinfection and with power supplied through a power cord in accordance with the disclosed architecture; and

FIG. 5 illustrates a schematic view showing a connection between one potential embodiment of the ultraviolet sterilization box of the present invention and an electronic device in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there exists a long felt need in the art for a sterilization box that can be used for sterilizing and disinfecting, for example, food items, bakery products, currency, keys, watches, sunglasses and other household items. There is also a long felt need in the art for a sterilization box that includes selectively-attachable germicidal lamps for sterilization of the stored items therein. Additionally, there is a long felt need in the art for a sterilization device that is lightweight, portable and capable of sterilizing items safely without posing a threat to a user. There is a long felt need in the art for a sterilization box that eliminates the use of harmful chemicals for disinfecting and sterilizing items. Moreover, there is a long felt need in the art for a sterilization box or device that can sterilize and irradiate non-planar surfaces of articles. Finally, there is a long felt need in the art for a sterilization box that provides a safe, convenient and reliable method to thoroughly sterilize and disinfect items in an efficient manner.

The present invention, in one exemplary embodiment, is a novel ultraviolet sterilization box. The ultraviolet sterilization box is configured for disinfecting household items such as food items, electronic items, wallets, keys, sunglasses and the like. The ultraviolet sterilization box further comprises: an internal chamber defined by a bottom surface and four walls extending vertically from the edges of the bottom surface; the internal chamber stores a plurality of items for disinfection; a lid pivotally-connected to one wall and is configured to cover the internal chamber; each of the walls, bottom surface and the lid have one or more UV-C lamps on interior surfaces for providing UV-C radiation from all directions to maximize the exposure of the articles within the internal chamber for sterilization. The UV-C lamps are automatically turned off after a predetermined period of time, and can be turned on manually using a control button or automatically through a sensor.

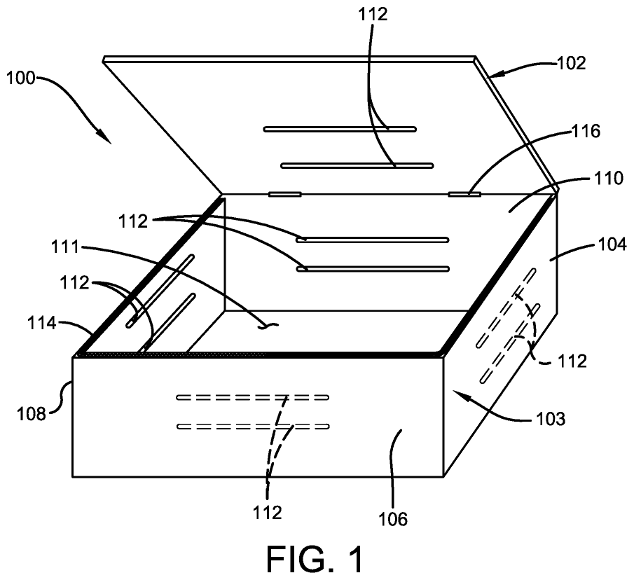

Referring initially to the drawings, FIG. 1 illustrates a perspective view of one potential embodiment of an ultraviolet sterilizer box of the present invention in accordance with the disclosed architecture. The ultraviolet sterilizer box 100 is generally cube-like or cuboidal-like in shape. The sterilizer box 100 is a portable disinfection unit configured for diffusing all kinds of fungi, bacteria, viruses and other microbes. The sterilizer box 100 can store and sterilize, for example, household items, food items and other everyday products. The sterilizer box or device 100 is lightweight and easy to carry for sterilizing items before and after use.

The sterilizer box 100 can be made from a waterproof flexible material, or from a hard material such as plastic or metal. The sterilizer box 100 can include an upper portion containing a lid 102 hinged to a base portion 103. The base portion 103 includes four walls 104, 106, 108, 110 extending vertically from the edges of a bottom surface 111. The walls, including two side walls 104, 108, a front wall 106, a back wall 110 and the bottom surface 111 (shown in FIG. 1) define an enclosed chamber configured to receive and store items for sterilization. Each wall is connected to the adjacent walls along the adjoining edges to form a complete enclosed space. The lid 102 is hinged along the top edge of the back wall 110 using hinges 116 to enable the lid 102 to pivotally move relative to the base portion. The lid 102 is configured to cover the enclosed space between the walls and can be fastened to the base portion using a fastener such as a zipper 114. Any conventional fastener such as snap button, hook and loop or magnetic fastener can be used in place of a zipper to fasten the lid 102 to any wall to completely cover the enclosed space.

The enclosed space is used for keeping the items for disinfection and can be accessed by opening the lid 102. The enclosed space can have one or more adjustable racks or trays to accommodate items at different levels for providing effective sterilization. The inner surfaces of the walls 104, 106, 108, 110, bottom surface 111 and the lid 102 can include germicidal lamps 112 to disinfect the stored items. The inner surfaces of each of the walls, lid and bottom surface can include two detachable germicidal lamps to provide adequate disinfection of the stored items. The germicidal lamps 112 can be configured to radiate ultraviolet (UV) radiation with a wavelength and intensity suitable for sanitization of any conventional article. The material of the box 100 can be substantially opaque, thereby prohibiting UV radiations from exiting the sterilizer box 100.

Each UV lamp 112 can be housed within an impact-resistant housing and can be selectively attached to the inner surfaces of the walls, lid, and bottom surface. To house the UV/germicidal lamps 112, the inner surfaces can have removable fasteners, including a pair of hooks that function to allow the UV lamps 112 to be affixed between the hooks. In one embodiment, the lid 102 can be transparent or translucent, thereby enabling a user to easily check if the UV lamps 112 are operational. Also, a transparent window can be present in any of the walls of the box 100 to enable the user to peer inside the box 100 to check the operational status of the UV-C lamps 112.

In one potential embodiment, the germicidal lamps 112 provide effective and environmentally friendly disinfection (sans chemicals) through the use of UV-C radiations at or about 254 nm wavelength. The germicidal lamps 112 have low mercury content and are ozone-free. The lamps 112 can be used for sterilization of any surface or objects. The UV-C lamps 112 can include power of up to 15 watts and can also be used for air and water sterilization. Additionally, the UV-C lamps 112 can use a built-in coil filament for sterilization.

Additionally, any commercially suitable source of UV radiation is contemplated for use with the sterilization box 100. In other embodiments, the UV lamps 112 can include cold cathode UV tubes, LEDs, and low, medium or high vapor mercury lamps. Suitable wavelengths that fall within the wavelength range of about 220 nm to about 300 nm can be used by the UV lamps 112 of the present invention. It is to be appreciated that UV lamps produce or emit light radiation from ultraviolet through visible to infrared radiation.

The UV-C lamps 112 are oriented radially inward towards the enclosed space so as to provide UV-C radiation from all directions to maximize the exposure of the articles within the enclosed space. The UV-C lamps on each inner surface can be spaced about two inches from each other. The UV-C lamps 112 can be provided laterally or longitudinally as per the preferences of a user.

It is to be appreciated that the number, size and position of each of the UV-C lamps 112 depends on the size and type of UV-C lamps used, the articles to be disinfected and the sizes and dimensions of the sterilization box 100. The UV-C lamps 112 can be disposed in any suitable location and oriented such that the articles stored within the box 100 are exposed to adequate amounts of UV-C radiation for sanitization.

The sterilization box 100 can be mounted to a wall or placed on any surface. It is understood that the size of the box 100 can vary, depending on the number and size of the articles to be disinfected. All commercially viable sizes are contemplated.

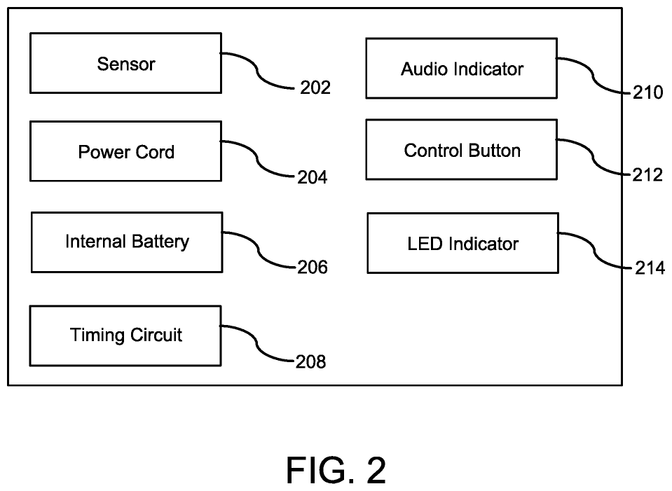

FIG. 2 illustrates a block diagram showing exemplary components of the ultraviolet sterilization box of the present invention in accordance with the disclosed architecture. A sensor 202 detects whether the lid of the sterilization box 100 is fastened to the base portion and in case the lid is unzipped or unfastened, the UV-C lamps are automatically turned OFF allowing a user to avoid contact with UV-C radiations. The sensor 202 can be an Infrared sensor or a near field communication (NFC) sensor. To provide disinfection using the UV-C lamps, a control button 212 is present to turn ON and/or OFF the UV-C lamps. The control button 212 can be a toggle button present on an exterior surface of any side wall of the box 100 to enable the user to operate UV-C lamps.

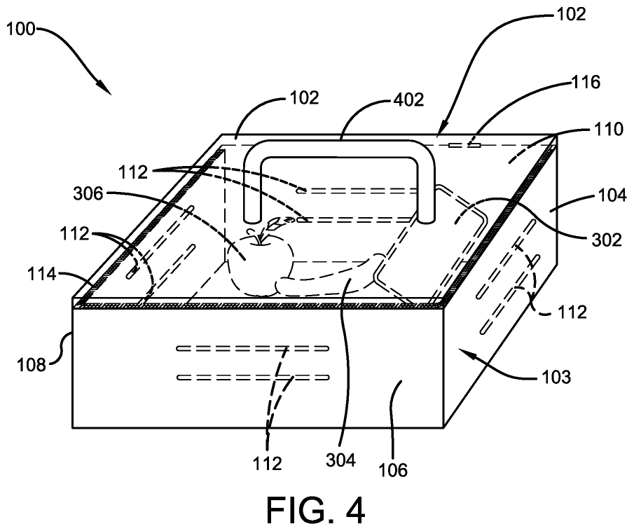

Electrical power can be provided to the UV-C lamps through regular power supply using a power cord 204. The power cord 204 can be plugged into a power outlet to provide power (FIG. 4). Alternatively, in some embodiments, an internal Li-ion battery 206 can provide power to the UV-C lamp, thereby allowing a user to disinfect items in conditions where a regular electrical power source is not present.

Advantageously, the disinfection box 100 of the present invention can automatically turn off the lamps after a predetermined period of time has elapsed. For example, in one scenario, the germicidal lamps are automatically turned off after ten minutes after the lid has covered the internal cavity of the box. A manual timer value can also be set to disinfect the items placed within the box 100. A variable timer knob can be present on an exterior surface of the box 100 that functions to allow for user adjustment of the UV illumination such that the user may selectively increase or decrease the time duration of UV illumination for each illumination cycle.

The disinfection box 100 features a timing circuit 208 that can be pre-configured with a timer value allowing the UV-C lamps to be active for the pre-configured timer value (i.e. predeterminable time period or duration). This functions for automatic turning off of the UV lamps.

To provide audio and visual notification of the sterilization process, the disinfection box 100 can have an audio indicator 210 that generates an alarm and an LED indicator 214 to illuminate an LED to indicate that the sterilization is complete and the items stored in the box are ready to be used. It should be appreciated that germicidal lamps of the disinfection box 100 of the present invention can also be used in combination with various timing devices to conserve electricity, as is known to those skilled in the art.

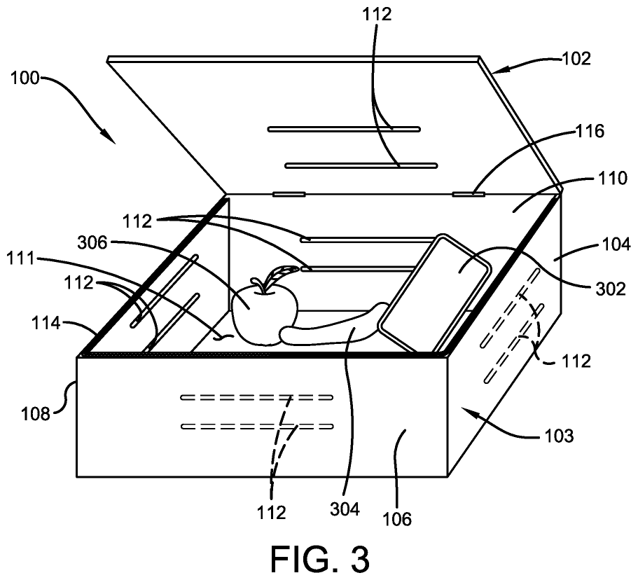

FIG. 3 illustrates a perspective view of items stored within one potential embodiment of an ultraviolet sterilizer box of the present invention for sterilization in accordance with the disclosed architecture. As illustrated, exemplary household items, edible items and other daily used items can be stored or placed within the box 100 allowing the UV-C lamps 112 to sterilize the items. As illustrated, an electronic device 302, a vegetable 304 and a fruit 306 are shown in the box 100 for sterilization. The items can be sterilized and microbes up to 99.9% can be killed in 5-10 minutes. The box 100 of the present embodiment is portable and foldable.

The box 100 provides for easy functioning of an UV-C sterilization chamber with varying capacity, such as 20 L and 40 L, and assures a heathier lifestyle with cleaner and more hygienic items. The UV-C light rays kill 99.99% of infectious disease-causing viruses and bacteria. UV-C irradiations from UV-C lights 112 provide 360-degree omnidirectional coverage inside the closed box. Additionally, the UV-C lights react on the DNA & RNA molecules of microorganisms and pathogens to provide hygienic items after sterilization. The box 100 of the present invention prohibits leakage of UV-C radiations and also prohibits production of ozone, therefore providing a safe device for users.

FIG. 4 illustrates a perspective view of the ultraviolet sterilization box of the present invention in a closed position with stored items for disinfection with power supplied through and electrical cord and outlet in accordance with the disclosed architecture. As stated earlier, the UV-C lamps 112 of the box 100 are automatically turned ON using the sensor that detects when the lid is in a closed position. The UV-C lamps 112 receive power from the direct power supply using the power cord 204. It should be understood that the timer circuit 208 can automatically stop the sterilization process even when the power cord 204 is supplying power to the UV-C lamps 112 for protection of the user using the box 100, and also for providing adequate sterilization of the items stored within the enclosed chamber of the box 100.

The box 100 can include a handle 402 to easily carry the box 100. The box 100 can also have a strap (not illustrated) to carry the box 100 on the shoulder by a user. The items 302, 304, 306 are effectively sterilized by the UV-C lamps 112 positioned on interior surfaces of all walls, bottom surface, and the lid of the box 100.

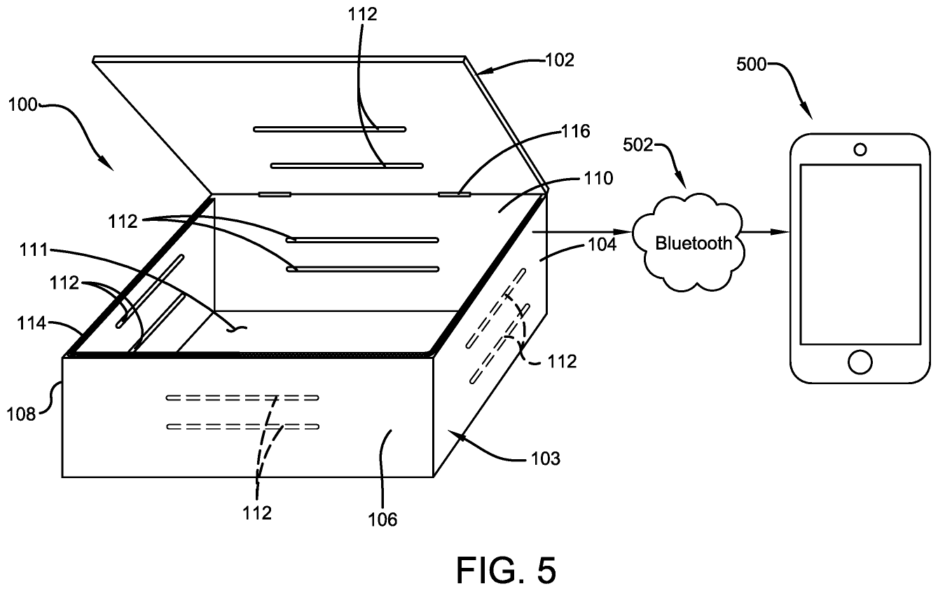

FIG. 5 illustrates a schematic view showing the wireless connection of one embodiment of the ultraviolet sterilization box of the present invention with an electronic device in accordance with the disclosed architecture. In the present embodiment, the sterilization box 100 has a wireless transceiver sewn into the surface of the box 100 allowing the box 100 to establish a wireless connection 502, such as a Bluetooth connection with a handheld device 500 such as a smartphone. Using the smartphone, the box 100 can be remotely turned ON/OFF and can be configured for sterilization. A companion smartphone app can be installed in the handheld device 500, thereby allowing the user to receive notifications of the sterilization status and of any malfunctioning of the box 100.

The UV-C lamps used in the present invention can include a length in the range of 3 inches to 12 inches, and the size of the UV-C lamps used in the box 100 can depend on the dimensions of the box 100. It should be appreciated that the present invention, in one potential embodiment, uses UV-C lamps which provide the greatest germicidal properties.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “ultraviolet sterilization box”, “sterilization box”, “disinfection box”, “UV-C sterilizer box”, and “box” are interchangeable and refer to the ultraviolet sterilization box 100 of the present invention.

Notwithstanding the forgoing, the ultraviolet sterilization box 100 of the present invention can be of any suitable size and configuration as is known in the art without affecting the overall concept of the invention, provided that it accomplishes the above-stated objectives. One of ordinary skill in the art will appreciate that the size, configuration, and material of the ultraviolet sterilization box 100 as shown in the FIGS. are for illustrative purposes only, and that many other sizes and shapes of the ultraviolet sterilization box 100 are well within the scope of the present disclosure. Although the dimensions of the ultraviolet sterilization box 100 are important design parameters for user convenience, the ultraviolet sterilization box 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A sterilization device comprising:
an enclosure for securing a plurality of objects to be sterilized;
said enclosure comprising a securable lid for enclosing the plurality of objects;
a plurality of UV lamps, wherein said plurality of UV lamps are selectively mounted to an interior surface of said enclosure and produce a light radiation in a range generally from about 220 nanometers to about 300 nanometers to perform a sterilization;
a timer for setting a predeterminable duration for the sterilization; and
said plurality of UV lamps automatically turn off after said predeterminable duration has expired.

2. The sterilization device of claim 1, wherein said securable lid is transparent.

3. The sterilization device of claim 1, wherein said securable lid is translucent.

4. The sterilization device of claim 1, wherein said plurality of UV lamps automatically turn OFF when said securable lid is opened.

5. The sterilization device of claim 1 further comprising an audible alarm when said plurality of UV lamps change from ON to OFF.

6. The sterilization device of claim 1 further comprising a transceiver for receiving a notification from a remote controller for activating said sterilization device.

7. The sterilization device of claim 6, wherein said remote controller comprises a Bluetooth connection with a mobile application.

8. The sterilization device of claim 1, wherein the range is from generally about 240 nanometers to about 260 nanometers.

9. The sterilization device of claim 8, wherein said light radiation is a UV-C radiation.

10. A sterilization device comprising:
an enclosure for securing a plurality of objects to undergo a sterilization;
said enclosure comprises a securable lid for enclosing the plurality of objects;
a plurality of UV lamps for producing a UV-C light radiation;
said plurality of UV lamps selectively mounted to each interior surface of said enclosure;
said UV-C light radiation in a range generally from about 240 nanometers to about 260 nanometers;
a timer for setting a predeterminable duration for the sterilization; and
said plurality of UV lamps automatically turn OFF after said predeterminable duration has expired.

11. The sterilization device of claim 10, wherein said securable lid is transparent.

12. The sterilization device of claim 10, wherein said securable lid is translucent.

13. The sterilization device of claim 11, wherein said plurality of UV lamps automatically turn OFF when said securable lid is opened.

14. The sterilization device of claim 13 further comprising an audible alarm when said plurality of UV lamps change from ON to OFF.

15. The sterilization device of claim 14 further comprising a transceiver for receiving a notification from a remote controller for activating said sterilization device.

16. The sterilization device of claim 15, wherein said remote controller comprises a Bluetooth connection with a phone app.

17. The sterilization device of claim 16 further comprising an electrical power cord for connecting with an electrical outlet to supply power to said plurality of UV lamps.

18. A sterilization device comprising:
an enclosure for securing a plurality of objects to undergo a sterilization, wherein said enclosure comprises a plurality of interior surfaces and a securable lid for enclosing the plurality of objects;
a plurality of UV lamps for producing a UV-C light radiation, wherein at least one of the plurality of UV lamps is selectively mounted to each of the plurality of interior surfaces of said enclosure, and further wherein said UV-C light radiation is in a range generally from about 220 nanometers to about 300 nanometers;
a timer for setting a predeterminable duration for the sterilization, wherein said plurality of UV lamps automatically turn OFF after said predeterminable duration has expired;
an audible alarm that emits an alarm when said plurality of UV lamps change from ON to OFF; and
a transceiver for receiving a notification from a remote controller for activating said sterilization device, wherein said remote controller comprises a Bluetooth connection with a phone app.

19. The sterilization of claim 18 further comprising an electrical power cord for connecting with an electrical outlet to supply power to said plurality of UV lamps.

20. The device of claim 19, wherein said range is from generally about 240 nanometers to about 260 nanometers.
